(19) United States
(12) Patent Application Publication
(10) Pub. No.: US 2026/0341756 A1
(43) Pub. Date: Oct. 29, 2026

(54) VENTED ENCLOSURE

(71) Applicant: Caterpillar Inc., Peoria, IL (US)

(72) Inventor: Leonardo Bergantim, St. Augustine, FL (US)

(73) Assignee: Caterpillar Inc., Peoria, IL (US)

(21) Appl. No.: 19/190,855

(22) Filed: Apr. 28, 2025

(51) Int. Cl. H01M 8/0444; B60P 3/00; G01N 33/00; H01M 8/04082; H01M 8/04746; H01M 8/2475

(52) U.S. Cl. CPC H01M 8/0444; B60P 3/00; G01N 33/0036; H01M 8/04201; H01M 8/04753; H01M 8/2475; H01M 2250/20

(57) ABSTRACT

An access compartment for a fuel system is disclosed and includes an enclosure defining a cavity and having an opening in a side wall thereof. Inside the cavity, there is a deflector which angles towards the opening. The deflector is configured to engage a gaseous material and direct it towards and through the opening, such that the gaseous mixture is no longer within the cavity. There is a sensor located on the deflector configured to measure a concentration of the gaseous mixture within the cavity. There is a plurality of fittings located inside the cavity and below the deflector, the fittings connecting a fuel source to a generator.

### TECHNICAL FIELD

The present disclosure relates generally to a vented enclosure, and more particularly to a passive and tip-resistant vented enclosure.

### BACKGROUND

Mobile power generation remains an effective and efficient way to provide power to areas that are remote, undergoing an emergency, or for temporary sites. Providing mobile power is generally done by trailering in a generator to where the need is and then removing it when no longer needed. Oftentimes, a diesel generator set is used, however propane, gasoline, and other fuels may also be used. Further, with developments in fuel cell technology, using hydrogen as a fuel source is becoming a more popular as a cleaner alternative to traditional fuels.

In some cases, the fuel and generator may be brought as a set, both contained within the trailer. However, it isn't uncommon for fuel to be available onsite, such that only the generator needs to be provided. Such a setup may remove the risk of hauling around large amounts of fuel. In such setups, the fuel onsite may be coupled directly to the generator on the trailer, minimizing time on setup and teardown. Due to the presence of combustible material, vented trailers are common to help mitigate any risks of concentrations of fuel from leaks and spills. However, vented trailers are not without their own drawbacks, such as vulnerability to weather changes or having security risks.

CN111682249A describes a hydrogen leakage safety protection system. The system of the '249 application includes two cavities, a discharge space stacked on top of an accumulation space. In the system, a lightweight gas rises from the accumulation space into the discharge space, and then from the discharge space out of the system. The system of the '249 application is a complex design which incorporates a fuel supply into the enclosure and presents multiple opportunities for hydrogen gas to accumulate.

### SUMMARY

In an aspect of the present disclosure, there is an access compartment for a fuel cell system, including an enclosure having a plurality of walls, including a first side wall having a first opening therethrough, and a cavity defined within the walls. There is also a deflector located within the cavity and having a first section angling upwards towards the first opening, the first section configured to engage a gaseous material/compound/fuel and direct the gaseous mixture towards the first opening. A plurality of fittings is located within the cavity below the deflector and configured to connect with at least one of a valve and a regulator and to communicate a gaseous material to the valve or regulator. A first sensor is connected to the deflector and configured to measure a concentration of the gaseous mixture. The deflector includes a first end section adjacent to the first opening, wherein the first sensor is located on the first end section, and the deflector connects with an end section of the first side wall. The first section includes an aperture and a lower face, and the first sensor includes a sensing end, and wherein the first sensor is mounted in the aperture such that the sensing end of is flush with the lower face. The first side wall is oriented in the vertical direction, and the first opening communicates with the ambient environment outside the enclosure. The enclosure further has a second side wall having a second opening therethrough, the second side wall opposite the first side wall, and the deflector further has a second section angling upwards towards the second opening, the second section configured to engage the gaseous mixture and direct it towards the second opening. The first sensor is connected to the first section and further including a second sensor connected to the second section and configured to measure a concentration of the gaseous mixture.

In another aspect of the present disclosure, there is a hydrogen fuel cell trailer system, including a storage compartment having an exterior surface and housing a first device chosen from a hydrogen storage device and a hydrogen fuel cell, an access compartment connected to the exterior surface and including an enclosure defining a cavity and having a first side wall having a first opening therethrough. There is a deflector located within the cavity and configured to direct a hydrogen fuel through the first opening. There is a hydrogen communication or transmission system, including a conduit extending from the storage compartment to the access compartment and configured to carry the hydrogen fuel, and a second device located within the cavity and below the deflector, chosen from a nozzle, a regulator, and a valve. There is a sensor coupled to the deflector and is configured to measure a concentration of the hydrogen mixture within the enclosure. The second device is a valve, the valve configured to be adjusted to a closed configuration if the concentration of the hydrogen mixture exceeds a threshold value. The deflector includes a first section angling upwards towards the first opening, the first section configured to engage the hydrogen mixture and direct it towards the first opening, the enclosure further includes a second side wall having a second opening therethrough, and the deflector further includes a second section angling upwards towards the second opening, the second section configured to direct the hydrogen mixture and direct it towards the second opening. There is a first sensor coupled to the first section and a second sensor coupled to the second section, each sensor configured to measure a concentration of the hydrogen mixture within the enclosure, the first sensor is adjacent to the first opening and the second sensor is adjacent to the second opening. The first side wall is oriented in the vertical direction, and the first opening communicates with the ambient environment outside the enclosure. The first device is a fuel cell and is coupled directly to the second device through the conduit such that the conduit is interrupted between the first device and the second device.

In yet another aspect of the present disclosure, a method is provided for a method of addressing hydrogen fuel emission in an access compartment of a fuel cell system, the method including Intercepting, with an angled deflector, a hydrogen fuel moving upwards within the access compartment, directing, using the angled deflector, the hydrogen mixture towards a vertical side wall of the access compartment, directing the hydrogen fuel across a sensor, sensing a concentration of the hydrogen fuel, and directing, using the angled deflector, the hydrogen fuel out of the access compartment through an opening in the side wall of the access compartment. The method also includes determining whether the concentration exceeds a threshold concentration and triggers an alarm and shuts off a flow of hydrogen gas to the access compartment if the concentration exceeds the threshold concentration.

Other features and aspects of this disclosure will be apparent from the following description and the accompanying drawings.

### BRIEF DESCRIPTION OF DRAWINGS

FIG. 1 is a perspective view depicting a portable generator system.

FIG. 2 is a perspective view of an access compartment from the portable generator system of FIG. 1.

FIG. 3 is a perspective view of a deflector plate from the access compartment of FIG. 2.

FIGS. 4A-4E are perspective views of the access compartment of FIG. 2 showing various stages of gas leakage within the access compartment.

FIG. 5 is a flowchart detailing the addressing of gas emissions within the access compartment.

### DETAILED DESCRIPTION

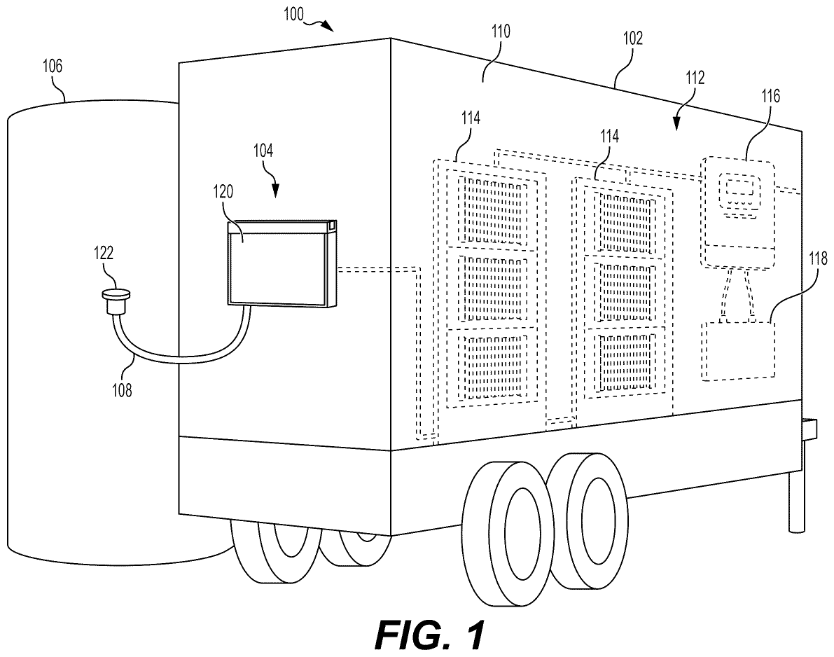

FIG. 1 shows a portable generator system 100. The system 100 includes a storage compartment 102 and an access compartment 104 and is shown attached to a fuel source 106 located away from the system 100. Fuel travels from the fuel source 106 and into a fuel line 108, entering the access compartment 104, and ultimately into the storage compartment 102, where it is used to generate electricity.

The storage compartment 102 includes an exterior surface 110, wherein a power generation system 112 is housed. The illustrated power generation system 112 includes multiple stacks of fuel cells 114 coupled to an inverter 116 and battery 118. The fuel cells 114 use fuel from the fuel source 106 to generate electricity. The inverter 116 is used to convert the DC output of the fuel cells 114 to AC current when necessary, however in other cases the output may be desired to be DC current, and the inverter 116 may not be necessary. The battery 118 is used to store unused energy generated by the fuel cells 114.

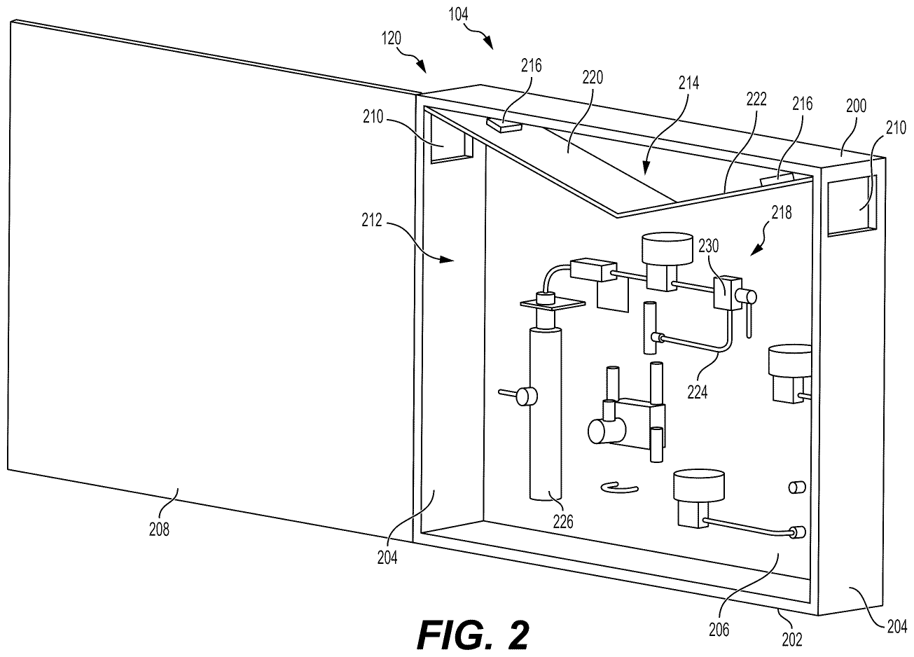

Connected to the exterior surface 110 is the access compartment 104, which includes an enclosure 120. The enclosure 120 houses various components used to transport and manipulate the fuel prior to it passing into the storage compartment 102, described further in FIG. 2. Coupled to the access compartment 104 through the fuel line 108 is the fuel source 106, illustrated as a fuel tank. The fuel source 106 has an access point 122 which regulates how much fuel may flow from the fuel source 106 to the access compartment 104.

Although the storage compartment 102 is illustrated as housing the power generation system 112, the storage compartment 102 may instead house the fuel source 106, or it may house both the power generation system 112 and fuel source 106. Further, although the power generation system 112 is depicted as a fuel cell system, in other embodiments it may be a traditional fuel burning generator which burns any number of fuels, such as but not limited to gasoline, diesel, propane, and natural gas. Additionally, the access compartment 104 is shown connected to the exterior surface 110, however it may be integrated into the storage compartment 102.

FIG. 2 shows the access compartment 104 from FIG. 1 in an open configuration. The access compartment 104 includes the enclosure 120, which includes a top wall 200, a bottom wall 202, a plurality of side walls 204, a back wall 206, and a door 208. The side walls 204 each include an opening 210 in a top section of each side wall 204 which provide direct access to the ambient environment from inside the enclosure 120. The walls, 200, 202, 204, and 206, and door 208 create a cavity 212.

Within the cavity 212 are a deflector 214, a plurality of sensors 216, and a fuel transmission system 218. The deflector 214 is located within the cavity 212 and is coupled to a top portion of the enclosure 120. The deflector includes a first section 220 and a second section 222. The first section 220 is connected to the second section 222 at connection point 224, and slopes upwards from the connection point 224 towards one of the openings 210. The second section 222 is shown as identical to the first section 220, and slopes upwards from the connection point 224 towards a separate opening 210.

The deflector 214 is shown having the sensors 216 connected thereto. One of the sensors 216 is shown connected to the first section 220, while another one of the sensors 216 is shown connected to the second section 222. The sensors 216 are shown shifted towards the openings 210 on each section, 220 and 222. The sensors 216 are configured to detect a concentration of a gas within the cavity 212, such as but not limited to hydrogen, gasoline, diesel, propane, and natural gas.

The fuel transmission system 218 is located below the deflector within the cavity and includes a conduit 224, a nozzle 226, a regulator 228, and a valve 230. The conduit 224 extends within the access compartment 104 as well as from the access compartment 104 to the storage compartment 102 and is configured to carry the fuel. The nozzle 226 is configured to be connected to the fuel source 106 and is where the fuel source 106 connects to the portable generator system 100. The regulator 228 is used to vary the flow and/or pressure of the fuel within the conduit 224 and to the fuel cells 114, such that they receive the fuel at the proper flow and/or pressure. The valve 230 restricts access to the fuel cells 114 from the fuel source 106. Each of the components of the fuel transmission system 218 are connected to one another through fittings.

In one embodiment, the fuel transmission system 218 may include one or more valves 230 between the nozzle(s) 226 and the other fuel transmission system 218 components. Accordingly, when the valve(s) 230 are actuated, fuel does not flow to the other fuel transmission system 218 components. The valve(s) 230 may be manually actuated or automatically actuated.

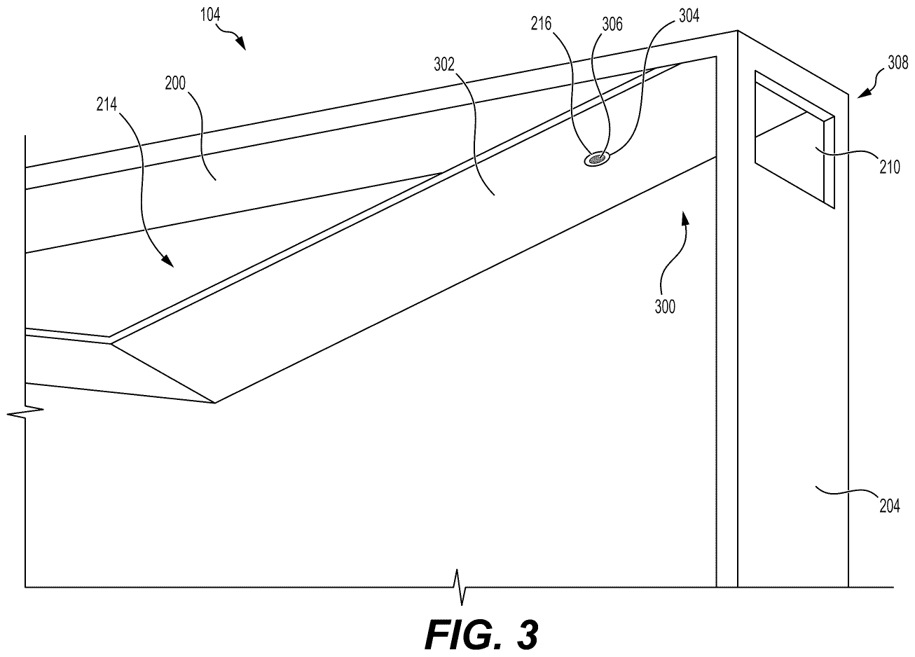

FIG. 3 is a view of the underside of the deflector 214 from the access compartment 104 of FIG. 2. The deflector 214 is shown having an end segment 300 adjacent to the opening 210 as well as an engagement surface 302 which is configured to engage the gas. As shown, the end segment 300 connects to an end section 308 of the side wall 204 at the opening 210. However, the end segment 300 may instead connect to an end section of the top wall 200 or may connect to both the top wall 200 and the side wall 204.

The deflector 214 further includes an aperture 304 extending through the engagement surface 302. The illustrated aperture 304 is within the end segment 300. The sensor 216 is situated within the aperture 304, such that a sensing end 306 is flush with the engagement surface 302. As illustrated, the sensor 216 is within the end segment 300. The remaining portions of the sensor 216 may be within the aperture 304, and/or behind the engagement surface 302.

The deflector 214 has an angular relationship with each of the the side walls 204, here illustrated as an acute angular relationship. Coupled with the location of the openings 210 in the end section 308 of the side wall 204, the access compartment 104 may be tilted towards either of the side walls 204 and still evacuate gas within the access compartment such that it does not accumulate.

Although the illustrated deflector 214 includes an engagement surface 302 which has a flat surface, the engagement surface 302 may have alternate surfaces which assist with funneling the gas towards the sensor 216. In one example, the engagement surface 302 may be semicircular, while in another example, the engagement surface 302 may be a plurality of sides coming together to form an angle at a topmost portion.

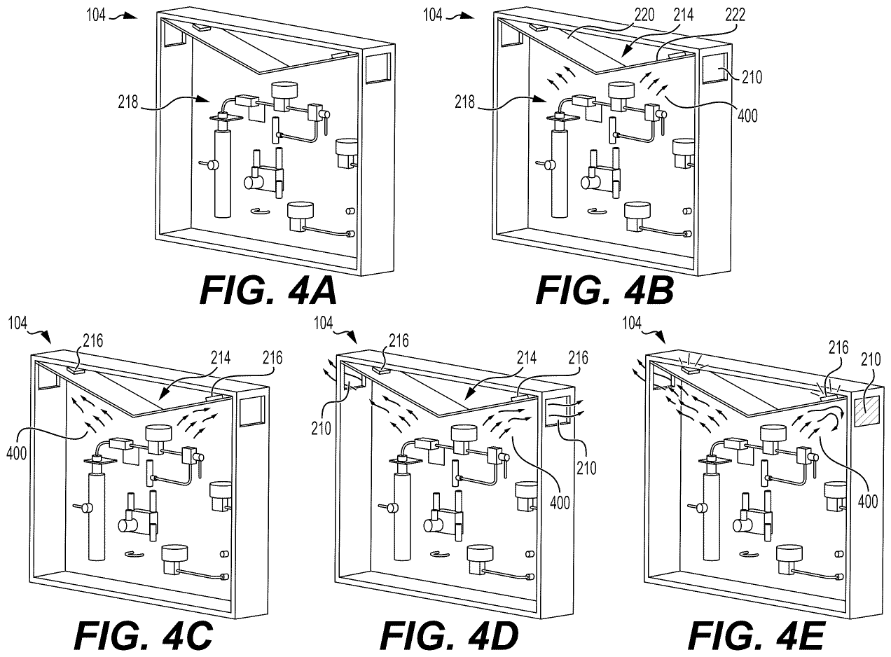

FIGS. 4A-4E are perspective views of the access compartment 104 of FIG. 2 showing various stages of gas leakage within the access compartment 104. In FIG. 4A, the fuel transmission system 218 is working as intended, and there is shown no gas leakage within the access compartment 104.

In FIG. 4B, a gas 400 is shown leaking from the fuel transmission system 218. The gas 400 rises and contacts the deflector 214. The gas 400 which contacts the first section 220 is deflected towards the opening 210 adjacent to the first section 220, while the gas 400 which contacts the second section 222 is directed towards the opening 210 adjacent to the second section 222.

In FIG. 4C, the gas 400 is shown flowing along the deflector 214 to the sensors 216. The sensors 216 periodically sense the concentration of the gas 400 flowing over them. Due to the gas 400 being lighter than air, the gas 400 collects along the deflector 214 as it rises upwards and outwards towards the openings 210, thereby concentrating the gas 400 at the deflector 214 and sensor 216. Accordingly, the sensed concentration of the gas 400 by the sensors 216 should be higher than the average concentration of the gas 400 within the access compartment 104.

In FIG. 4D, the gas 400 is shown flowing along the deflector 214, away from the sensors 216, to, and ultimately through, the openings 210. Accordingly, the gas 400 is passively evacuated from the access compartment 104 such that accumulation of the gas 400 within the access compartment 104 is minimized. As shown, the gas 400 exiting the access compartment 104 through the openings 210 goes directly to the atmosphere and is not routed through another chamber or structure. However, in other embodiments, the openings 210 may direct the gas 400 to another chamber or structure.

In FIG. 4E, the gas 400 is shown to be leaking at an increased rate. As shown, the sensors 216 have been triggered due to the concentration of the gas 400 sensed by the sensors 216 exceeding a threshold level. In this case, the sensor 416 is shown triggering an alert to bring attention to the condition. In some cases, the alert may instead be or further include shutting off the flow of fuel, such as by using a valve 230, such as a shutoff valve, to or from the access compartment 104.

Even in the case of a small leak which wouldn't normally trigger the sensors 216, if one of the openings 210 is partially or fully obstructed, a portion of the rising gas 400 that cannot escape the access compartment 104 may become trapped near the sensor 216. In such a situation, the gas 400 may continue to displace the ambient air in the area, and accumulate, driving the concentration of the gas 400 to be higher near the sensor 216. Similarly, the concentration may be sufficient to exceed the threshold level, triggering the sensor(s) 216 and a response.

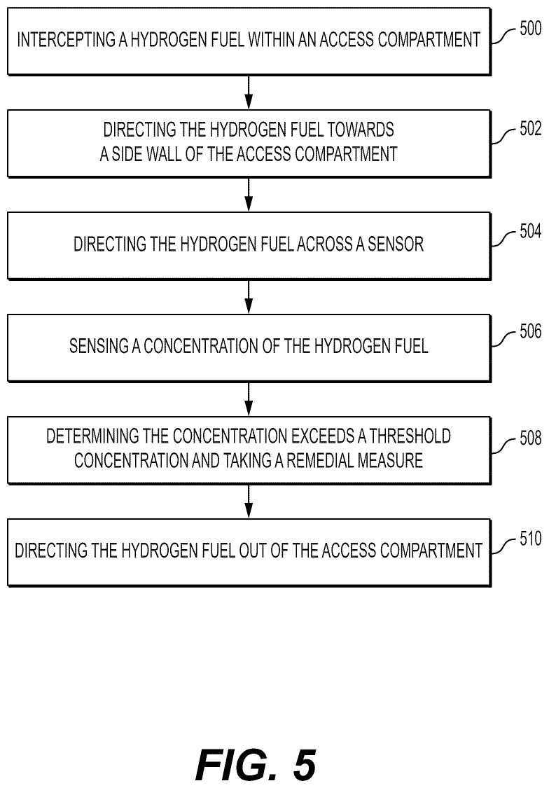

FIG. 5 is a flow chart detailing a method of using a vented access compartment 104. Although detailed as a using multiple openings 210, an access compartment 104 with a single opening 210 may be used following substantially the same method.

At step 500, a hydrogen fuel is intercepted within the access compartment 104. During this step, rising hydrogen gas within the cavity 212 contacts the deflector plate 220. The deflector plate 220 is a solid structure and is sealed to the sides of the access compartment 104. Accordingly, the rising gas cannot pass through or around the deflector plate 220 to move past it within the access compartment 104.

At step 502, the gas is directed towards the side wall 204 of the access compartment 104. Since the gas cannot move around the deflector plate 220, the angle of the deflector plate 220 allows the gas to rise along the slanted bottom surface towards the opening(s) 210 in the access compartment 104. The deflector plate 220 illustrated previously splits the rising gas, such that the gas contacting the first section 220 is deflected to one opening 210, while gas contacting the second section 222 is deflected to a separate opening 210 opposite the other opening 210. (reword)

At step 504, the gas, on its way towards the openings 210, is directed by the deflector plate 220 across a sensor 216. The sensing surface of the sensor 216 is located generally flush with the bottom surface of the deflector plate 220 such that the gas flows directly across the sensing surface and does not become trapped by the sensor 216. In the examples disclosed

At step 506, the sensor 216 measures the concentration of the gas flowing over the sensing surface. Generally, since any gas within the access compartment 104 is constantly directed out of the access compartment 104, in the case of a leak there should be a constant flow of gas over the sensor 216, such that there is no collection of stagnant gas within the access compartment 104. The sensor 216 is constantly detecting the concentration of the gas over time.

At step 508, sensor 216 determines if the concentration of the gas exceeds a threshold concentration. For example, the threshold concentration for hydrogen may be as low as 2% to 4%. As the sensor 216 is taking readings, it compares them against the threshold concentration. In the case of a leak and/or blockage of the opening(s) 210, the concentration at the sensor 216 may rise above the predetermined threshold. In a case where the sensor 216 measures a concentration above the threshold, step 508 further includes taking remedial measures. The remedial measures may include the sensor 216 or device collecting the sensor 216 reading triggering an auditory, visual, and/or haptic alarm. The sensor 216 or device collecting the sensor 216 reading may also or instead restrict or shut off one or more components of the fuel transmission system 218. For example, in one embodiment, when the threshold is passed, an alert may be sent to an operator and a valve within the fuel transmissions system 218 may be closed, preventing the flow of the gas within the fuel transmission system 218.

At step 510, the gas is directed out of the access compartment 104. The angle of the deflector plate 220 allows the gas to further rise along the slanted bottom surface towards the opening(s) 210 in the access compartment 104. The deflector plate 220 extends towards the top side of the opening(s) 210, such that the gas is guided directly to and through the opening(s) 210, out of the access compartment 104, and directly into the atmosphere.

### INDUSTRIAL APPLICABILITY

The above disclosure is a vented access compartment 104 for a portable generator system 100 which uses a fuel that may give off an undesirable vapor. The access compartment 104 may hold one or more components that are prone to leakage within the component or at one or more fittings, such that leakage from those components may be monitored by sensors 216 and directed out of the access compartment 104 and into the atmosphere.

Accordingly, the deflector 220 and openings 210 within the access compartment 104 minimizes the chances of an accumulation of undesirable vapor accumulation within the access compartment 104. Further, in the case of an accumulation, the sensors 216 may alert and operator or shut off a flow of fuel to or within the access compartment 104 such that any problems may be remedied. Further, the design of the deflector 220 and openings 210 allow the venting function of the access compartment to work effectively despite substantial tilting of the access compartment 104, such that the portable generator system 100 may be set up on uneven terrain. As such, the system 100 is more reliable and requires less checkups to ensure it is working as intended.

While aspects of the present disclosure have been particularly shown and described with reference to the embodiments above, it will be understood by those skilled in the art that various additional embodiments may be contemplated by the modification of the disclosed machines, systems, and methods without departing from the spirit and scope of the disclosure. Such embodiments should be understood to fall within the scope of the present disclosure as determined based upon the claims and any equivalents thereof.

What is claimed is:

1. An access compartment for a fuel cell system, comprising:
an enclosure having:
a plurality of walls, including a first side wall having a first opening therethrough; and
a cavity defined within the walls;
a deflector located within the cavity and having a first section angling upwards towards the first opening, the first section configured to engage a gaseous material/compound/fuel and direct the gaseous mixture towards the first opening;
a plurality of fittings located within the cavity below the deflector and configured to connect with at least one of a valve and a regulator and to communicate a gaseous material to the valve or regulator; and
a first sensor connected to the deflector and configured to measure a concentration of the gaseous mixture.

2. The access compartment of claim 1, wherein:
the deflector includes a first end section adjacent to the first opening, wherein the first sensor is located on the first end section; and
the deflector connects with an end section of the first side wall.

3. The system of claim 1, wherein the first section includes an aperture and a lower face, and the first sensor includes a sensing end, and wherein the first sensor is mounted in the aperture such that the sensing end of is flush with the lower face.

4. The system of claim 1, wherein the first side wall is oriented in the vertical direction, and the first opening communicates with the ambient environment outside the enclosure.

5. The system of claim 1, wherein:
the enclosure further has a second side wall having a second opening therethrough, the second side wall opposite the first side wall; and
the deflector further has a second section angling upwards towards the second opening, the second section configured to engage the gaseous mixture and direct it towards the second opening.

6. The system of claim 5, wherein the first sensor is connected to the first section and further including a second sensor connected to the second section and configured to measure a concentration of the gaseous mixture.

8. A hydrogen fuel cell trailer system, comprising:
a storage compartment having an exterior surface and housing a first device chosen from the group consisting of: a hydrogen storage device and a hydrogen fuel cell;
an access compartment connected to the exterior surface and including:
an enclosure defining a cavity and having a first side wall having a first opening therethrough; and
a deflector located within the cavity and configured to direct a hydrogen fuel through the first opening; and
a hydrogen communication or transmission system, including:
a conduit extending from the storage compartment to the access compartment and configured to carry the hydrogen fuel; and
a second device located within the cavity and below the deflector, chosen from the group consisting of: a nozzle, a regulator, and a valve.

9. The system of claim 8, further including a sensor coupled to the deflector, the sensor configured to measure a concentration of the hydrogen mixture within the enclosure.

10. The system of claim 9, wherein the second device is a valve, the valve configured to be adjusted to a closed configuration if the concentration of the hydrogen mixture exceeds a threshold value.

11. The system of claim 8, wherein:
the deflector includes a first section angling upwards towards the first opening, the first section configured to engage the hydrogen mixture and direct it towards the first opening;
the enclosure further includes a second side wall having a second opening therethrough; and
the deflector further includes a second section angling upwards towards the second opening, the second section configured to direct the hydrogen mixture and direct it towards the second opening.

12. The system of claim 11, further including a first sensor coupled to the first section and a second sensor coupled to the second section, each sensor configured to measure a concentration of the hydrogen mixture within the enclosure.

13. The system of claim 12, wherein the first sensor is adjacent to the first opening and the second sensor is adjacent to the second opening;

14. The system of claim 8, wherein the first side wall is oriented in the vertical direction, and the first opening communicates with the ambient environment outside the enclosure.

15. The system of claim 8, wherein the first device is a fuel cell and is coupled directly to the second device through the conduit such that the conduit is interrupted between the first device and the second device.

16. A method of addressing hydrogen fuel emission in an access compartment of a fuel cell system, the method comprising:
Intercepting, with an angled deflector, a hydrogen fuel moving upwards within the access compartment;
directing, using the angled deflector, the hydrogen mixture towards a vertical side wall of the access compartment;
directing the hydrogen fuel across a sensor,
sensing a concentration of the hydrogen fuel; and
directing, using the angled deflector, the hydrogen fuel out of the access compartment through an opening in the side wall of the access compartment.

17. The method of claim 16, further including determining whether the concentration exceeds a threshold concentration.

18. The method of claim 17, further including triggering an alarm if the concentration exceeds the threshold concentration.

19. The method of claim 17, further including shutting off a flow of hydrogen gas to the access compartment if the concentration exceeds the threshold concentration.

20. The method of claim 19, wherein the threshold concentration is 2%.
